(19) United States
(12) Patent Application Publication
(10) Pub. No.: US 2004/0082554 A1
(43) Pub. Date: Apr. 29, 2004

(54) Neuroactive steroid derivatives and method of use

(76) Inventors: David F Farb, Brookline, MA (US); Ghazaleh Sadri-Vakili, Boston, MA (US); Robert Christopher Pierce, Boston, MA (US); David W Johnson, Kennebunk, ME (US); Terrell T Gibbs, Jamaica Plain, MA (US)

Correspondence Address: FISH & RICHARDSON PC, 225 FRANKLIN ST, BOSTON, MA, 02110, US

(21) Appl. No.: 10/415,791

(22) PCT Filed: Nov. 6, 2001
(86) PCT No.: PCT/US01/43975

(51) Int. Cl. A61K 31/56

(52) U.S. Cl. 514/169

(57) ABSTRACT

The invention relates to neuroactive steroid compounds that are useful in modulating CNS effects, diseases or disease symptoms. The invention also relates to use of the compounds in methods of treating or preventing disease or disease symptoms, and methods of modulating or mediating CNS effects or processes.

### STATEMENT REGARDING FEDERALLY SPONSORED RESEARCH

This invention was supported in part by grant number NM49469 from the National Institutes of Health (NIH). The U.S. Government may have certain rights in the invention.

### BACKGROUND

Diseases of the central nervous system (CNS) are very complex in nature, invoking, for example, combinations of organs (e.g., the brain), nerve systems and pathways, ligand-receptor interactions, and molecular communication processes. Because of their complex mechanisms of action as well as the wide impact of these diseases on health and well being, including cognition, consciousness, behavior and neuromuscular effects, the study of the disease processes and development of new therapeutic approaches to management of these diseases is of high interest.

### SUMMARY

The invention relates to neuroactive steroid compounds that are useful in modulating CNS effects, diseases or disease symptoms. The invention also relates to use of the compounds in methods of treating or preventing disease or disease symptoms, and methods of modulating or mediating CNS effects or processes.

The invention relates to a method of treating neurologic disease or disease symptoms in a subject by administration of an effective amount of a neuroactive steroid. The neurologic neurologic disease or disease symptom can be mediated by NMDA, one mediated by NMDA-induced increase in dopamine release, one mediated by glutamatergic transmission, one involving negative modulation of glutamatergic transmission, or any of drug addiction, schizophrenia, or Parkinson's disease. The neuroactive steroid useful in the methods herein include those that cross the blood-brain barrier, such as pregnanolone hemisuccinate.

Another aspect of the invention relates to any method of treating disease or disease symptoms delineated herein wherein the neurologic disease or disease symptom is treated by cognitive enhancement, including those wherein the neurologic disease or disease symptom is amnesia or other memory-impairment. In another aspect the methods are those wherein the neuoractive steroid affects an impaired subject but essentially does not affect a normal subject, that is, the agent is more efficacious for treating impaired subjects, and leads to lesser or diminished undesireable side effects. Another aspect is the methods of treating disease or disease symptoms herein wherein the neuoractive steroid is pregnenolone hemisuccinate.

The invention also relates to a method of inducing acute anesthesia in a subject comprising administration of an effective amount of a neuroactive steroid, including that wherein the subject is conscious and can communicate while under the effect of acute anesthesia. For example, this application can be useful in situations where a surgical procedure is necessary, however, it may be preferable that the patient be able to communicate with the medical team while the procedure is ongoing.

The invention also relates to a method of treating a disorder, the disorder being characterized by excessive NMDA levels in a subject (e.g. drug addiction or schizophrenia) comprising administration of an effective amount of a neuroactive steroid (e.g., pregnanolone hemisuccinate).

The invention also relates to a method of treating a disorder potentiated by NMDA in a subject comprising administration of an effective amount of a neuroactive steroid (e.g., pregnenolone hemisuccinate).

In another aspect, the invention is any of the methods delineated herein, wherein the treating is essentially void of side effects associated with bizarre or psychotic behavior, that is, is essentially void of behavior such as uncontrolled shaking, circling, psychotic hallucinations, and the like.

The invention also relates to a method of enhancing memory function in a subject comprising administration of an effective amount of a neuroactive steroid (e.g., pregnenolone hemisuccinate) and a method of enhancing acetylcholine release in the brain of a subject comprising administration of an effective amount of a neuroactive steroid (e.g., pregnenolone hemisuccinate).

In one aspect, the invention relates to methods of treating or preventing pain (e.g., method of providing analgesia) to a subject in need thereof including administration of an effective amount of neuroactive steroid derivative (e.g., 3α5βHS, PACME) or composition thereof. Certain neuroactive steroid derivatives are shown to be more efficacious than morphine in treating or mediating pain.

In one aspect, the compounds and methods delineated herein are neuroactive steroids that are capable of crossing the blood-brain barrier. In one aspect, the compounds and methods delineated herein are neuroactive steroids that are capable of carrying a negative charge, including upon metabolism, (e.g., anionic in form or corresponding protic or salt form).

The compounds of the invention are useful in treating or preventing disease, including, but not limited to, neurologic and psychiatric disease, disease symptoms, or disorders. Such disorders, include, for example, drug craving, drug addiction (e.g., cocaine, morphine), compulsive disorders, schizophrenia, amnesia, memory loss, dementia (e.g., Alzheimer's disease (AD), substance-induced, vascular, early onset familial), epilepsy, hypoxic neuronal damage, excitotoxicity, Parkinson's disease, stroke, ischemia, motor control disorders, spinal cord injury, and pain (e.g., nociceptive pain). The compounds and methods thereof are useful as anxiolytics, sedatives, hypnotics, analgesics, and anesthetics. As anxiolytics, the compounds herein offer alternate side-effect profiles from current therapies, such as benzodiazepines or barbituates.

Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The invention can provide several advantages over the existing methods of treatment. For example, the compounds of the invention can have several chemical and pharmacological advantages useful in treating disease or disease symptoms. These advantages can include both chemical stability and pharmacological stability, as well as potency, different resistance profiles, different tolerance profiles, different selectivity profiles, and decreased side-effects. The invention also envisions veterinary uses for the treatment of disease in animals (e.g., dogs, cats, or horses). Thus, a subject as described herein includes these animals as well as humans.

Other features and advantages of the invention will be apparent from the following detailed description, drawings, and from the claims.

### DESCRIPTION OF DRAWINGS

FIG. 1. Chemical structure of pregnanolone hemisuccinate (3α5βHS).

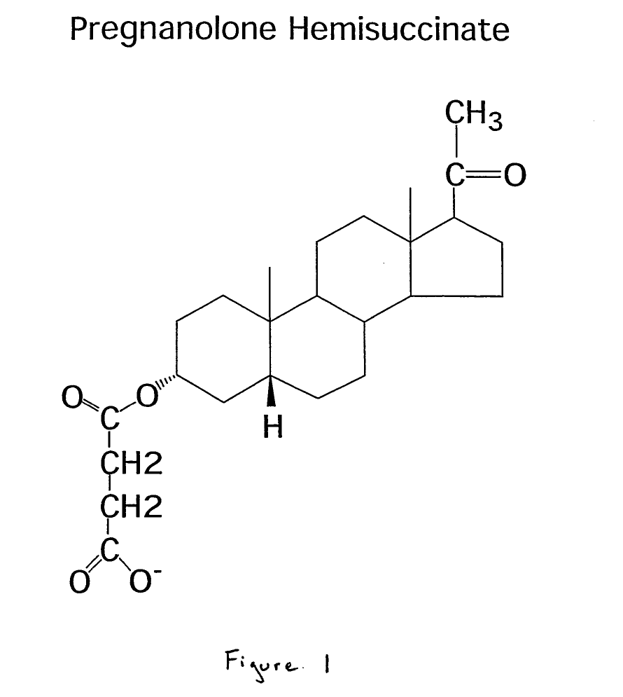

FIG. 2. 3α5βHS rapidly enters the brain following systemic injection. Male rats were injected with 3α5βHS (10 mg/kg, i.p). 3α5βHS was extracted from whole brain, and analyzed via HPLC ESI-MS. Data represent the mean nmol/g 3α5βHS in whole brain obtained from 3 animals±SEM. Note that 3α5βHS enters the brain within 10 min after injection and declines to near baseline levels by 60 min. Data represent the average nmol/g pregnanolones of 3 animals±SEM.

FIG. 3. (A) Location of microdialysis probes in the striatum. (B) Location of microinjection sites in the striatum. The lines represent the placement of the microdialysis probes and the circles represent the tip of the microinjection cannulae. The numbers on each section represent millimeters from bregma. The brain sections are from the atlas of Paxinos and Watson (1997).

FIG. 4. 3α5βHS iinhibits behavioral activity induced by NMDA in the rat striatum. (A) 3α5βHS (1, 10 mg/kg) or DMSO (0.5 μl/g) were administered i.p. 10 min prior to the microinjection of NMDA (10 μg/ml) or sterile saline into the rat striatum. Following microinjection, animals were placed in behavioral boxes and horizontal activity was monitored at 5 min intervals for one hour. (B) Bar graph represents the horizontal count at 10 min from FIG. 3A to clearly demonstrate levels of significance. * Denotes significant increase in horizontal activity compared to the saline group while # represents significant difference between treatment groups as compared to control (p<0.05, Fisher's LSD). The animals treated with NMDA+vehicle show an increase in horizontal activity as compared to the saline group. However, animals treated with NMDA+3α5βHS (1 or 10 mg/kg) show a decrease in horizontal activity compared to the NMDA+vehicle group, Number of animals used in microinjection experiments were 6-8 per group.

FIG. 5. D-AP5 inhibits 1 mM NMDA-evoked release of extracellular dopamine in the rat striatum in a dose-dependent manner. (A) Rats were injected with 100% DMSO (0.5 μl/g, i.p.) immediately after collection of the sample at time 0. After a 10 min waiting period, the perfusion medium was switched from aCSF to a 1 mM NMDA solution with D-AP5 (100 μM or 200 μM) dissolved together in aCSF. After collection of the next 20 min sample, the perfusion medium was switched back to aCSF and 5 more samples were collected. Data are mean±SEM for extracellular concentrations of dopamine, presented as percentages of baseline values. The infusion of 1 mM NMDA+D-AP 5 (100 μM or 200 μM) solution started at time 10 and lasted for 20 min (indicated by horizontal bar). (B) Data represents peak times for 20 and 40 min from FIG. 4A. NMDA-induced increases in extracellular dopamine release are inhibited by D-AP5 in a dose-dependent manner at 20 and 40 min. * Denotes significant difference from baseline levels of dopamine while # represents significant difference between treatment groups compared to aCSF(p<0.05, Fisher's LSD). For the in vivo microdialysis experiments the number of animals used were 4-6 per group.

FIG. 6. 3α5βHS inhibits 1 mM NMDA-evoked release of extracellular dopamine in the rat striatum in a dose-dependent manner. (A) Rats were injected with either 100% DMSO (0.5 μl/g, i.p.) or 3α5βHS (0.01, 1, 5, 10, 20 mg/kg, i.p.) immediately after collection of the sample at time 0. After a 10 min waiting period, the perfusion medium was switched from aCSF to a 1 mM NMDA solution dissolved in aCSF. After collection of the next 20 min sample, the perfusion medium was switched back to aCSF and 5 more samples were collected. Data are mean±SEM values for extracellular concentrations of dopamine, presented as percentages of baseline values. The infusion of 1 mM NMDA solution started at time 10 and lasted for 20 min (indicated by horizontal bar). (B) Data represents peak times for 20 and 40 minutes from FIG. 5A. 3α5βHS inhibits NMDA-induced dopamine release at a 5, 10 or 20 mg/kg dose. * Denotes significant difference from baseline levels of dopamine for the 5, 10 and 20 mg/kg dose while # represents significant difference between treatment groups compared to control (p<0.05, Fisher's LSD). (C) Dose-response curve for 3α5βHS inhibition of NMDA-induced dopamine release at 20 min. Data points: percentage change in NMDA-induced striatal dopamine release with an i.p. injection of 3α5βHS (mean of 4-6 experiments). Error bars: S.E.M. Curve is a nonlinear least-squares fit to the Michealis-Menton equation; EC50 for 3α5βHS is 4 mg/kg. For the in vivo microdialysis experiments the number of animals used were 4-6 per group.

FIG. 7. Pregnanolone has no influence on NMDA induced striatal dopamine release. (A) Rats were injected with pregnanolone (160 μg/kg, i.p.) at time 0. After a 10 min waiting period, the perfusion medium was switched from aCSF to a 1 mM NMDA solution dissolved in aCSF. After the collection of the next 20 min sample, the perfusion medium was switched back to aCSF and 5 more samples were collected. Data are mean±SEM (bars) values for extracellular concentrations of dopamine, presented as percentages of baseline values. The infusion of 1 mM NMDA solution started at time 10 and lasted for 20 min (indicated by horizontal bar). (B) Data represents peak times for 20 and 40 min from FIG. 6A. There is no difference between the vehicle treated and pregnanolone treated groups as seen in the bar graph. For the in vivo microdialysis experiments the number of animals used were 4-6 per group.

FIG. 8. 3α5βHS inhibits the initiation of behavioral sensitization to cocaine. 3α5βHS (10 mg/kg) or saline (0.9%) were administered i.p. 10 min prior to each of 7 daily injections of cocaine (15 mg/kg, i.p.). Following the injections, animals were placed in behavioral boxes and activity was monitored in 10 min intervals for 2 hours. Data represents horizontal activity monitored over 120 min post-injection. (B) Data represent the 50 min time point from FIG. 7A. There is a significant increase in the horizontal count in the saline/cocaine group on day 7 compared to the 3α5βHS/cocaine group, indicating an inhibition of behavioral sensitization to cocaine by the steroid. * Denotes significant increase in horizontal activity on day 7 compared to day 1, while # indicates significant difference between the 3α5βHS/cocaine and saline/cocaine group on day 7, (p<0.05, Fisher's LSD). Number of animals used in cocaine sensitization experiments were 5 per group.

FIG. 9. Results of the dose-dependent antinociceptive effect of 3α5βHS in the tail flick test. Mice were injected with 3α5βHS (5, 10 or 15 mg/kg, i.p.). Following the injection the mice were tested for every 10 min for 120 min. The latency to remove the tail from the light source was used as a measure of analgesia. 3α5βHS increased the latency to remove the tail from the light source dose-dependently in a manner similar to that of morphine. The values are mean+SEM for 10-15 mice for each group. Data were analyzed using a one-way ANOVA followed by Fisher's LSD.* Denotes significance compared to control.

FIG. 10. The analgesic effect of PACME in the tail flick test. Mice were injected with PACME (5, 10 or 15 mg/kg). Following the injection the mice were tested for every 10 min for 120 min. The latency to remove the tail from the light source was used as a measure of analgesia. PACME increased the latency to remove the tail from the light source dose-dependently in a manner similar to that of morphine. Data were analyzed using a one-way ANOVA followed by Fisher's LSD. The values are mean±SEM for 10-15 mice for each group.* Denotes significance compared to control.

FIG. 11. Chemical structure of pregnenolone hemisuccinate (PEHS).

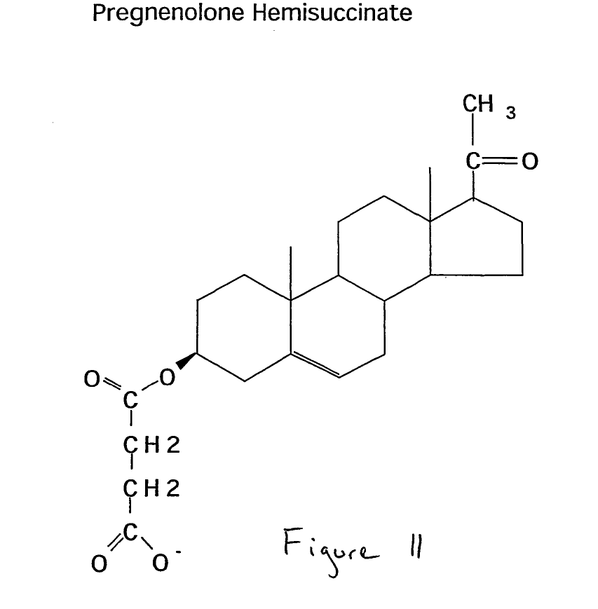

FIG. 12. Chemical structure of pregnanolone carboxy methyl ether(PACME).

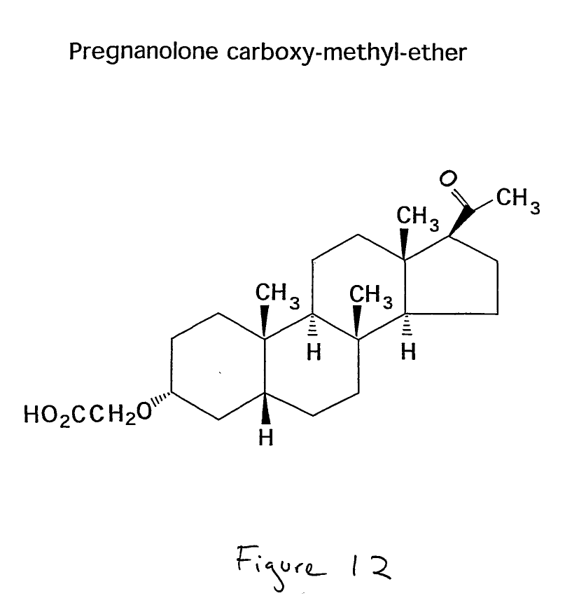

Like reference symbols in the various drawings indicate like elements.

### DETAILED DESCRIPTION

A neuroactive steroid derivative is any steroid compound, or compound having essentially a steroid core structure, that affects a neurological event, process, pathway, communication process, and the like. The compounds of this invention are commercially available (e.g., from Steraloids Inc., Wilton, N.H.) or can be synthesized using conventional techniques. Advantageously, these compounds are conveniently synthesized from readily available starting materials. In general, the compounds of the formulae described herein are conveniently obtained via standard organic chemistry synthesis methods, including those methods illustrated in the schemes and the examples herein. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995); and subsequent editions thereof.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a subject (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

The compounds of this invention can be modified by appending appropriate functionalities to enhance selective biological properties. Such modifications are known in the art and include those which increase biological penetration into a given biological compartment (e.g., blood, lymphatic system, central nervous system), increase oral bioavailability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, butyrate, citrate, camphorate, camphorsulfonate, ethanesulfonate, formate, fumarate, glycolate, heptanoate, hexanoate, hydrochloride, hydrobromide, 2-hydroxyethanesulfonate, lactate, maleate, malonate, methanesulfonate, 2-naphthalenesulfonate, nicotinate, nitrate, palmoate, pectinate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, can be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products can be obtained by such quaternization.

The heterocyclic compounds of the formulae delineated herein can be administered to a patient, for example, in order to treat disease or disease symptoms. The heterocyclic compounds can, for example, be administered in a pharmaceutically acceptable carrier such as physiological saline, in combination with other drugs, and/or together with appropriate excipients.

As the skilled artisan will appreciate, lower or higher doses than those recited above can be required. Specific dosage and treatment regimens for any particular patient will depend upon a variety of factors, including the activity of the specific compound employed, the age, body weight, general health status, sex, diet, time of administration, route of administration, frequency of administration, rate of excretion, drug combination, the severity and course of the disease, condition or symptoms, the patient's disposition to the disease, condition or symptoms, and the judgment of the treating physician.

Pharmaceutical compositions of this invention include a compound of the formulae described herein or a pharmaceutically acceptable salt thereof; an additional agent, such as a CNS agent, and any pharmaceutically acceptable carrier, adjuvant or vehicle. Alternate compositions of this invention include a compound of the formulae described herein or a pharmaceutically acceptable salt thereof; and a pharmaceutically acceptable carrier, adjuvant or vehicle. Such compositions can optionally include additional therapeutic agents, including, for example an additional agent such as a pain relief agent (e.g., nonsteroidal anti-inflammatory drug (NSAID)), an additional CNS agent, or an antinausea agent. The compositions delineated herein include the compounds of the formulae delineated herein, as well as additional therapeutic agents if present, in amounts effective for achieving a modulation of the levels of NMDA or acetylcholine, or for enhancing cognitive function.

The term “pharmaceutically acceptable carrier or adjuvant” refers to a carrier or adjuvant that can be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be advantageously used to enhance delivery of compounds of the formulae described herein.

The pharmaceutical compositions of this invention can be administered orally, parenterally, by inhalation spray, topically, rectally, nasally, buccally, vaginally, subdermally, transmucosally, or via an implanted reservoir, preferably by oral administration or administration by injection. The pharmaceutical compositions of this invention can contain any conventional non-toxic pharmaceutically-acceptable carriers, adjuvants or vehicles. In some cases, the pH of the formulation can be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional, intraperitoneally, and intracranial injection or infusion techniques.

The pharmaceutical compositions can be in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and/or suspensions. Other commonly used surfactants such as Tweens or Spans and/or other similar emulsifying agents or bioavailability enhancers which are commonly used in the manufacture of pharmaceutically acceptable solid, liquid, or other dosage forms can also be used for the purposes of formulation.

The pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient can be suspended or dissolved in an oily phase combined with emulsifying and/or suspending agents. If desired, certain sweetening and/or flavoring and/or coloring agents can be added.

The pharmaceutical compositions of this invention can also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, bees wax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention can also be topically applied to the lower intestinal tract by rectal suppository formulation or in a suitable enema formulation. Topically applied transdermal patches are also included in this invention.

The pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

Dosage levels of between about 0.001 and about 100 mg/kg body weight per day, alternatively between about 0.5 and about 75 mg/kg body weight per day, or any range in which the lower number is between 0.001 and 99.9 inclusive, and the upper number is between 0.002 and 100 inclusive and is higher than the lower number, mg/kg body weight per day, of the compounds described herein are useful in a monotherapy and/or in combination therapy for the prevention and treatment of disease. Typically, the pharmaceutical compositions of this invention will be administered from about 1 to about 6 times per day (e.g., at 10 mg-1000 mg/dose; or any range in which the lower number is an integer between 10 and 99.9 inclusive, and the upper number is an integer between 11 and 1000 inclusive that is higher than the lower number) or alternatively, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

When the compositions of this invention include a combination of a compound of the formulae described herein and one or more additional therapeutic or prophylactic agents, both the compound and the additional agent should be present at dosage levels of between about 10% to 100%, and more preferably between about 10% to 80% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

The compounds of this invention can contain one or more asymmetric centers and thus occur as racemates and racemic mixtures, single enantiomers, individual diastereomers and diastereomeric mixtures. All such isomeric forms of these compounds are expressly included in the present invention. The compounds of this invention can also be represented in multiple tautomeric forms (see illustration), in such instances, the invention expressly includes all tautomeric forms of the compounds described herein. All such isomeric forms of such compounds are expressly included in the present invention. All crystal forms of the compounds described herein are expressly included in the present invention.

Assessment of the activity of the compounds delineated herein can be performed using a variety of protocols known in the art, including those exemplified in the representative examples herein. Such methods include in vitro and in vivo models, and can utilize direct or indirect measurements efficacy. Reagents and instrumentation associated with these techniques and protocols are commercially available from a variety of vendors and sources, including those specifically listed herein.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

The invention will be further described in the following examples. It should be understood that these examples are for illustrative purposes only and are not to be construed as limiting this invention in any manner.

### EXAMPLES

Materials and Methods

Subjects. Male Sprague-Dawley rats (225-300 g) from Charles River Laboratories (Wilmington, Mass.) were initially housed in shoebox cages (2 rats/cage) and were provided with food and water ad libitum. The cages were kept in a temperature-controlled room with a 12-hour light/dark cycle. All experiments were performed during the light cycle.

Chemicals. All steroids were purchased from Steraloids Inc. (Wilton, N.H.) and were dissolved in 100% DMSO for systemic injections. Cocaine was a gift from the National Institute of Drug Abuse. NMDA and D-AP5 were purchased from Sigma (St. Louis, Mo.) and dissolved in artificial cerebrospinal fluid (aCSF) (145 mM NaCl; 2.7 mM KCl; 1.2 mM CaCl₂; 1.0 mM MgCl₂; 0.2 mM ascorbate; 5.0 mM glucose; pH 7.4).

Determination of Pregnanolone Hemisuccinate Tissue Levels. Rats were housed for one week prior to initiation of experiments. On the day of the experiment, rats were divided into three groups based on their scheduled time of sacrifice (10, 20 and 60 minutes post injection) and injected with 3α5βHS (10 mg/kg, i.p.). Rats were anesthetized with isoflurane, and decapitated at the designated time points. Brain tissue was rapidly removed, dissected, and stored at −80° C. until assayed.

For each rat 0.6 to 2 g of tissue was analyzed for 3α5βHS; the larger tissue amounts were required for samples originating from the 60 min time-point. Pregnanolone hemiglutarate (PAHG) (1 μl of a 2.7 mM solution in ethanol) was added to tissue samples for use as an extraction recovery standard. Tissues were homogenized in 2 ml of 0.25 M NaOH with a sonic dismembrator. Potentially interfering lipids were extracted from the alkaline homogenates with 15 ml of n-hexane in three aliquots. Homogenates were then acidified by the addition of 2.5 ml of 2.5 M H₂PO₄. 3α5βHS and PAHG were then extracted from the homogenates with the aid of a sonic dismembrator using three 5 ml aliquots of 10% ethyl acetate in hexane.

Extraction aliquots were combined, diluted with 10 ml of n-heptane, and loaded onto a conditioned 500 mg silica SepPak® column (Waters Corp; Milford, Mass.). Columns were washed with 5 ml hexane, and steroidal analytes were eluted with 3 ml of a 1:1 mixture of acetone:hexane. The eluant was evaporated to dryness under vacuum and reconstituted in 49 μl of CH₃OH and 1 μl of 50 mM ammonium acetate containing 2.5 pmol pregnenolone hemisuccinate (PEHS) for use as an instrument standard.

Chromatographic resolution was achieved using a HP 1100 HPLC running a mobile phase of 75% CH₃OH, 25% 50 mM ammonium acetate (pH 3) at a flow rate of 100 μl/min through a Hypersil BDS C18 column (2.1×150 mm) (Alltech Associates; Deerfield, Ill.). Column effluent was directed into a HP 5989x electrospray mass spectrometer operating in the negative ion mode and running a sheath liquid of 50 mM NH₄OH at 5 μl/min. The mass spectrometer was set to monitor the deprotonated ions of the analyte and two standards (3α5βHS m/z=417.2, PAHG m/z=431.3, PEHS m/z=415.2).

Sample values of 3α5βHS were normalized to the optimal response of the instrument standard, and then determined by comparing to a concurrently run, external standard curve. 3α5βHS values were then corrected for extraction efficiency, which was determined by monitoring PAHG recovery.

Microinjection Experiments. Rats were anesthetized with sodium pentobarbital (50 mg/kg, i.p.) and placed in a stereotaxic apparatus (David Kopf Instruments). A rostro-caudal incision was then made to expose the dorsal surface of the skull. Two bilateral holes were drilled above the striatum (+1.0 mm A/P, +3.0 mm M/L, −3.0 mm D/V relative to bregma, Paxinos and Watson, 1997) and guide cannulae (9 mm, 24 gauge) were implanted and fastened to the skull using screws and dental cement. All rats were housed in individual cages following surgery. Three days post surgery rats were habituated to the photocell apparatus (AccuScan Instruments; Columbus, Ohio) for two hours. On the day of the experiment rats were rehabituated for one hour prior to the start of the experiment. An injection of 100% DMSO (0.5 μl/g, i.p.) or 3α5βHS (1 or 10 mg/kg, i.p.) was administered prior to microinjection. Ten minutes following the injection, obturators were removed from the guide cannulae and replaced by a 33-gauge stainless steel injection needle that extended 2 mm below the tip of the guide cannulae into the striatum. Bilateral infusions of NMDA (10 μg/μl) or 0.9% saline were made over 2 min at a volume of 0.5 μl per side into the striatum. The injectors remained in the striatum for one min following the injection to ensure that the compounds had diffused from the site of injection. The animals were returned to the behavior boxes and activity was monitored continuously for 60 min.

In vivo Microdialysis. Prior to surgery the animals were anesthetized with sodium pentobarbital (50 mg/kg, i.p) and placed in a stereotaxic apparatus (David Kopf Instruments). A rostro-caudal incision was then made to expose the dorsal surface of the skull. A hole was drilled in the skull above the striatum with a stereotaxic drill (+0.5 mm A/P and +2.9 mm M/L relative to bregma, (Paxinos and Watson, 1997). Two other holes were drilled in the skull where screws were secured. A dialysis guide cannula (CMA Microdialysis; Acton, Mass.) was lowered 3 mm ventrally and was then secured to the skull with dental cement. All rats were housed in individual cages following surgery.

No less than two days after surgery, the rats were briefly anesthetized with isoflurane to facilitate the removal of the dummy from the guide cannula and insertion of a microdialysis probe (CMA 10, dialysis membrane length of 2 mm of polycarbonate, with a molecular weight cut off of 20 kD) into the striatum. Animals were allowed to recover for 15 min prior to the initiation of the experiment following probe insertion. Artificial cerebrospinal fluid (aCSF) was pumped through the dialysis probe at a rate of 2 μl/min. Twelve 20 min samples were collected over 240 min and immediately stored at −20° C. The first six 20 min samples were collected to establish the baseline concentration of dopamine. Following samples 1-6, the rats received an injection of either vehicle (DMSO 0.5 μl/g, i.p.) or 3α5βHS (0.01, 1, 5, 10 or 20 mg/kg, i.p.) before the collection of sample 7. At this point the perfusate for the DMSO group was changed from aCSF to one of the following solutions: NMDA (1 mM) or NMDA (1 mM) plus D-AP5 (100-200 μM). The perfusate for the steroid group was switched from aCSF to NMDA (1 mM). After collection of sample 7, the perfusate was switched back to aCSF and 5 more 20 min samples were collected. Upon termination of the experiment, samples were stored at −80° C. until analysis.

All samples were analyzed using high pressure liquid chromatography (HPLC) with electrochemical detection (ESA; Chelmsford, Mass.). The mobile phase consisted of 50 mM Na₂HPO₄, 20 mM citric acid, 1.5 mM heptanosulfonic acid, 0.1 mM EDTA, and 5% methanol at pH 4.0. The dopamine was separated on a 15 cm reverse phase C-18 column (Alltech; Deerfield, Ill.) and oxidized at a potential of 175 mV. Dopamine concentrations per sample were normalized to an external standard curve and plotted as percentage change from baseline.

Cocaine Sensitization. Rats were habituated to the photocell apparatus (AccuScan Instruments; Columbus, Ohio) for two hours and administered saline (i.p.) one day prior to the start of the experiment. On the first day of the experiment, rats were rehabituated to the behavior boxes for one hour prior to the start of the experiment. Saline (0.9%) or 3α5βHS (10 mg/kg) was injected systemically 10 min prior to an injection of cocaine (15 mg/kg, i.p.). Horizontal activity was measured for 2 hours after every cocaine injection. Saline-cocaine or 3α5βHS-cocaine were administered daily on 7 consecutive days.

Histology. Upon completion of the microinjection and microdialysis experiments the animals were given an overdose of sodium pentobarbital (100 mg/kg, i.p.) and were perfused intracardially with 0.9% saline followed by 10% formalin. The brains were removed and coronal sections (100 μm) were taken at the level of the striatum using a Vibratome (Technical Products International; St. Louis, Mo.). The sections were stained with Cresyl violet. Cannulae placement and potential neurotoxicity were determined by an individual who was unaware of prior treatments.

### Example 1

### Systemically Administered 3α5βHS Enters the CNS

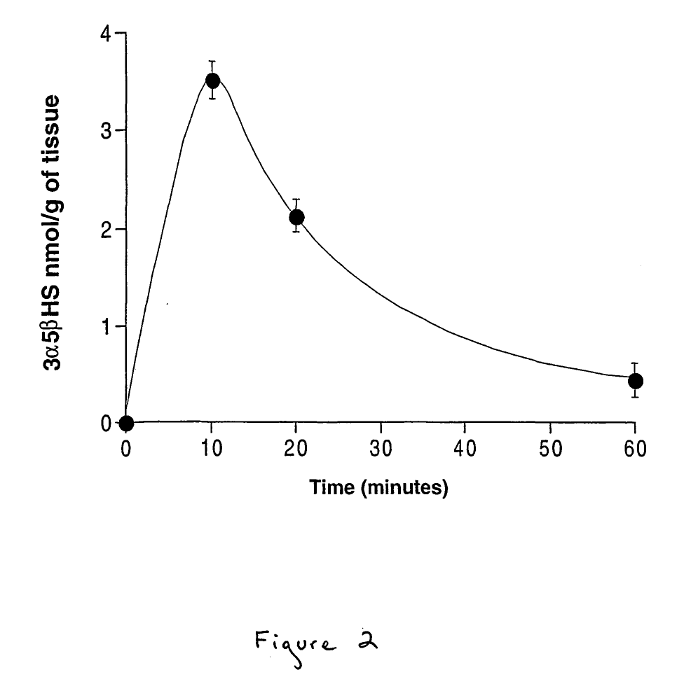

To assess the rate of appearance and decay of the steroid in the brain, rats were given an injection of 3α5βHS (10 mg/kg, i.p.) and sacrificed at 10, 20, and 60 min post injection. 3α5βHS was extracted from whole brain, and analyzed via HPLC ESI-MS. 3α5βHS was detected in the brain within 10 min (FIG. 2). Steroid levels reached 3.5 nmol/g of brain tissue at 10 min post injection and declined to 0.5 nmol/g of brain tissue by 60 min. While the level of 3α5βHS peaked 10 min post-injection, the concentration in the brain remained elevated 20 min following injection and declined to near-baseline levels by 60 min. These results were utilized in determining the time frame of steroid injection for microinjection and microdialysis experiments.

Histological Examination

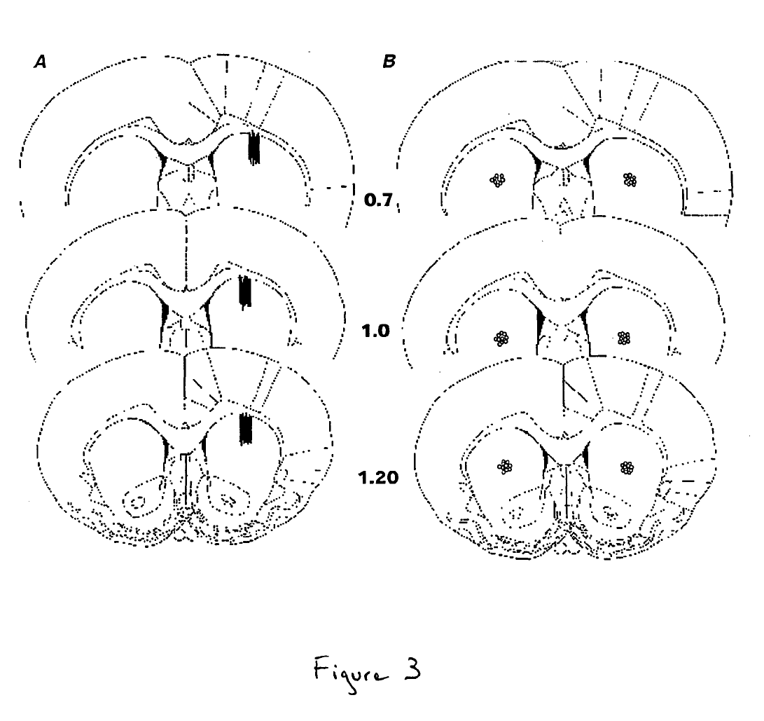

Histological results (FIG. 3) indicate placement of the dialysis probes and the microinjection cannulae within the dorsal striatum. The brain sections were examined under a microscope and determined to be free of NMDA-induced neurotoxicity. Some expected mechanical damage was detected secondary to probe placement and cannulation.

### Example 2

### The Effect of 3α5βHS on the Behaviorial Hyperactivity Induced by Intra-striatal NDMA

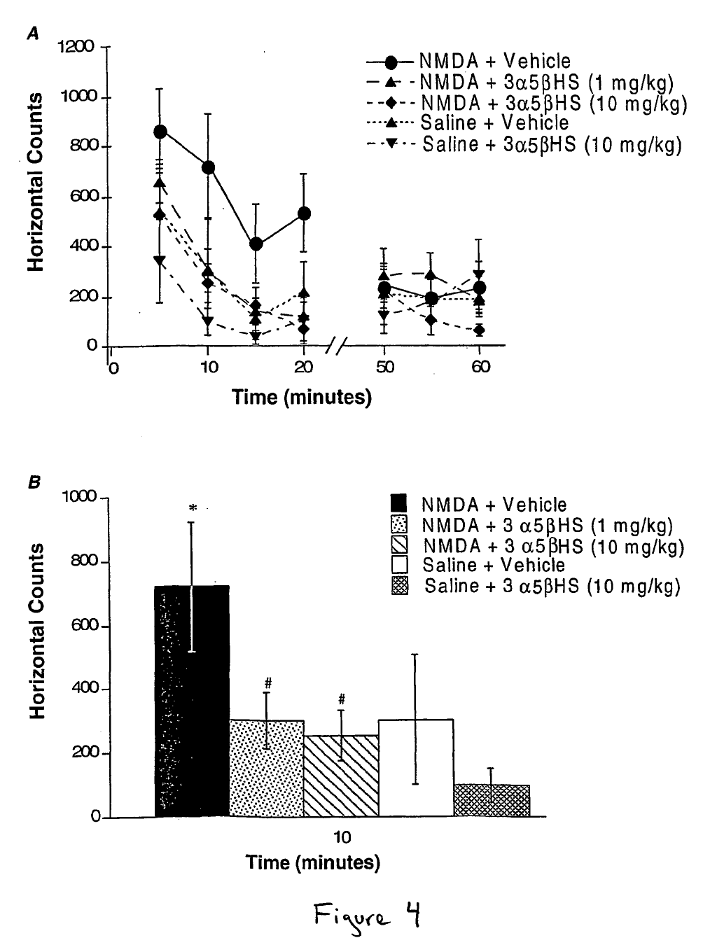

Rats were administered with one of two doses of 3α5βHS (1 mg/kg or 10 mg/kg) or 100% DMSO (1 mg/kg) intraperitoneally 10 min prior to intra-striatal microinjection of NMDA or saline. Horizontal activity was measured in 5 min blocks for 60 min immediately following microinjection. The data were analyzed with a mixed factors analysis of variance (ANOVA) with repeated measures over time in FIG. 4A. The analysis revealed a significant main effect of time [F(11,352)=6.63, p<0.0001] and a marginally significant main effect of treatment [F(44, 32)=2.54, p<0.058]. There was no significant treatment×time interaction. Subsequent pairwise analyses (Fisher's LSD) revealed a significant increase in horizontal activity following NMDA microinjection at 5, 10, 15 and 20 min. The data in FIG. 4B represent the 10 min time point from FIG. 4A and was analyzed using a one-way ANOVA. While this analysis revealed a marginally significant main effect [F(4,32)=2.577, p<0.0562], post-hoc (Fisher's LSD) analyses indicated that the NMDA-induced behavioral activation was blocked by 3α5βHS (1 & 10 mg/kg) (FIG. 4B). 3α5βHS (10 mg/kg) did not induce sedation, reflected by no difference between the saline+vehicle and the saline+3α5βHS groups at any time point (FIG. 4).

### Example 3

### The Effect of 3α5βHS on NMDA-induced Dopamine Release

Basal extracellular levels of striatal dopamine were measured in 40 rats via in vivo microdialysis. The average (±standard error of the mean) extracellular dopamine values was 2.0±0.8 nmol/20 μl.

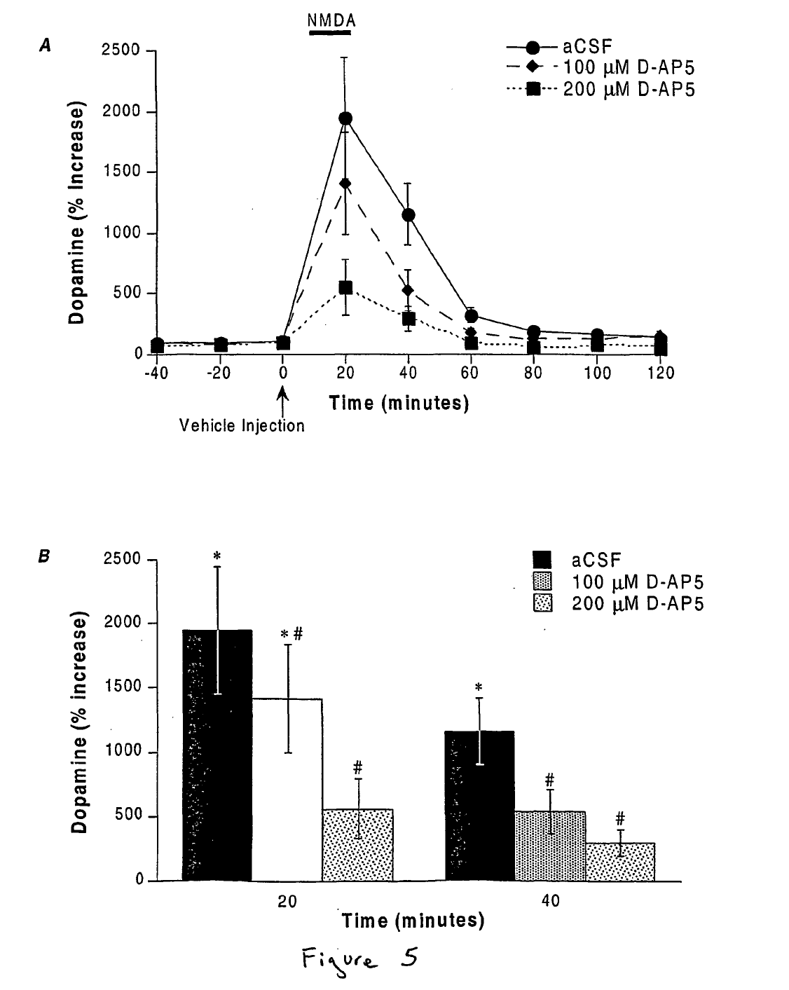

The data presented in FIG. 5 indicate that NMDA-induced striatal dopamine release is blocked by the NMDA antagonist, D-AP5. The data are presented as percent baseline, with baseline defined as the average of the four samples collected prior to administration of NMDA. The complete time course of the microdialysis data are presented in FIG. 5A. The data collected 20 and 40 min following the intra-striatal perfusion of NMDA is presented in FIG. 5B. The complete time-course was analyzed with a mixed factors ANOVA with repeated measures over time. The results of this analysis revealed significant main effects of treatment [F(2,12)=3.83; p<0.05] and time [F(8,96)=16.57, p<0.0001] as well as a significant treatment×time interaction [F(16,96)=1.84, p<0.036]. Data in FIG. 5B were analyzed with a one-way ANOVA, which revealed no significant main effect [F(2,12)=1.785, p<0.2096] at 20 min while the analysis at time 40 proved to be significant [F(2,12)=3.875, p<0.0503]. Subsequent pairwise analyses (Fisher's LSD) showed that there was a significant increase in extracellular dopamine in the striatum 20 and 40 min following perfusion of NMDA through the microdialysis probe. The NMDA-induced increase in dopamine was attenuated in a dose-dependent manner by the co-perfusion of NMDA with 100 μM or 200 μM D-AP5 (FIG. 5).

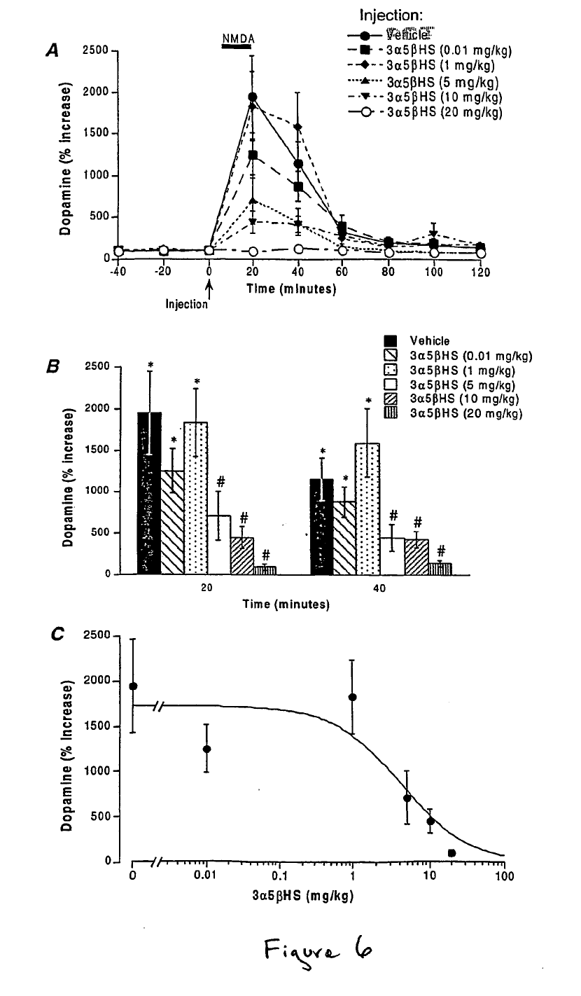

Similar to D-AP5, systemic administration of 3α5βHS (0.01, 1, 5, 10 or 20 mg/kg) decreased NMDA-induced dopamine release dose-dependently (FIG. 6). The data are presented as percent baseline, with baseline defined as the average of the 4 samples collected prior to administration of NMDA. The complete time course of the microdialysis data are presented in FIG. 6A. The data collected 20 and 40 min following the intra-striatal perfusion of NMDA is presented in FIG. 6B. The complete time-course was analyzed with a mixed factors ANOVA (repeated measures over time). The results of this analysis revealed significant main effects of treatment [F(5,22)=5.52; p<0.0019] and time [F(8,176)=37.5, p<0.0001] as well as a significant treatment×time interaction [F(40,176)=4.16, p<0.0001]. The data in FIG. 6B were analyzed with a one-way ANOVA and the analysis revealed significant main effects at 20 min [F(5,22)=4.455, p<0.0059] and 40 min [F(5,22)=5.16, p<0.0028]. Subsequent pairwise analyses (Fisher's LSD) showed that there was a significant increase in extracellular dopamine in the striatum 20 and 40 min following perfusion of NMDA and that this effect was blocked by 3α5βHS at the following doses, 5, 10 and 20 mg/kg. FIG. 6C illustrates the dose-dependent effect of 3α5βHS on NMDA-induced dopamine release. The NMDA-induced increase in dopamine was dose-dependently decreased by 3α5βHS with an EC50 of 4 mg/kg.

### Example 4

### The Effect of Pregnanolone on NMDA-induced Dopamine Release

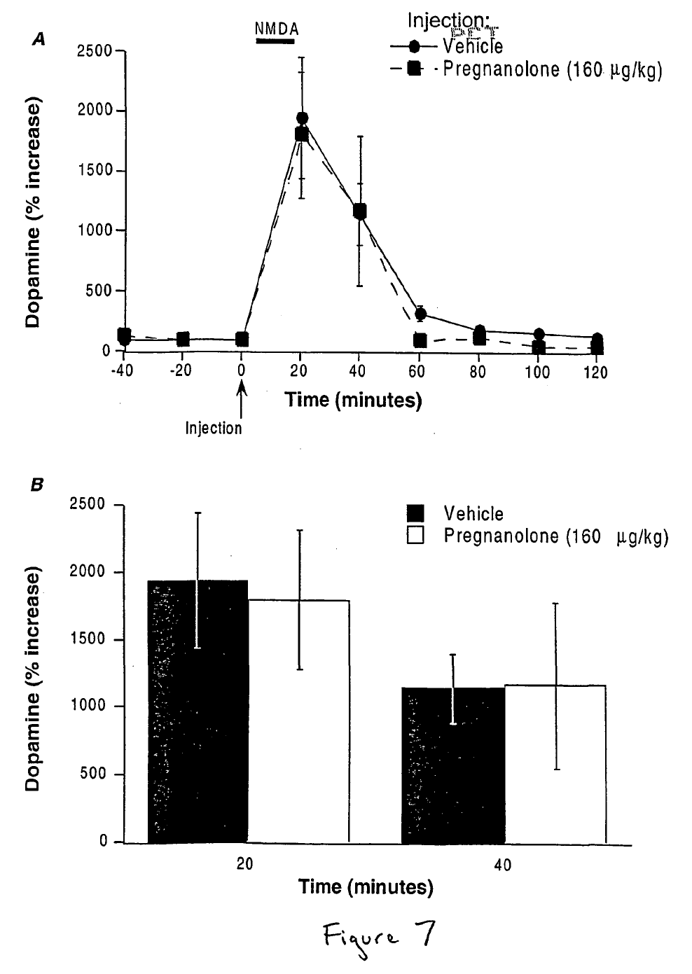

While systemic injections of 3α5βHS (10 mg/kg) decreased NMDA-induced dopamine release, pregnanolone, the major metabolite of 3α5βHS, had no effect on the increase in striatal dopamine induced by NMDA (FIG. 7). The data are presented as percent baseline, with baseline defined as the average of the 4 samples collected prior to administration of NMDA. The complete time course of the microdialysis data are presented in FIG. 7A. The data collected 20 and 40 min following the intra-striatal perfusion of NMDA are presented in FIG. 7B. The complete time-course was analyzed with a two-way mixed factors ANOVA with repeated measures over time. The results of this analysis revealed a significant main effect of time [F(8,56)=17.0; p<0.0001] but no significant main effect of treatment nor a significant time×treatment interaction. The data in FIG. 7B were analyzed by one-way ANOVA and the analysis revealed no significant main effects at 20 min [F(2,10)=3.18, p<0.0853] or 40 min [F(2,10)=1.723, p<0.2276]. Moreover, we found that there was no difference in the levels of pregnanolones present in the brain following 3α5βHS (10 mg/kg) [10.9±2.8 pmol/g tissue] compared to saline injections [6.3±1.8 pmol/g tissue] at 20 min post injection (data not shown).

### Example 5

### The Effect of 3α5βHS on Cocaine Sensitization

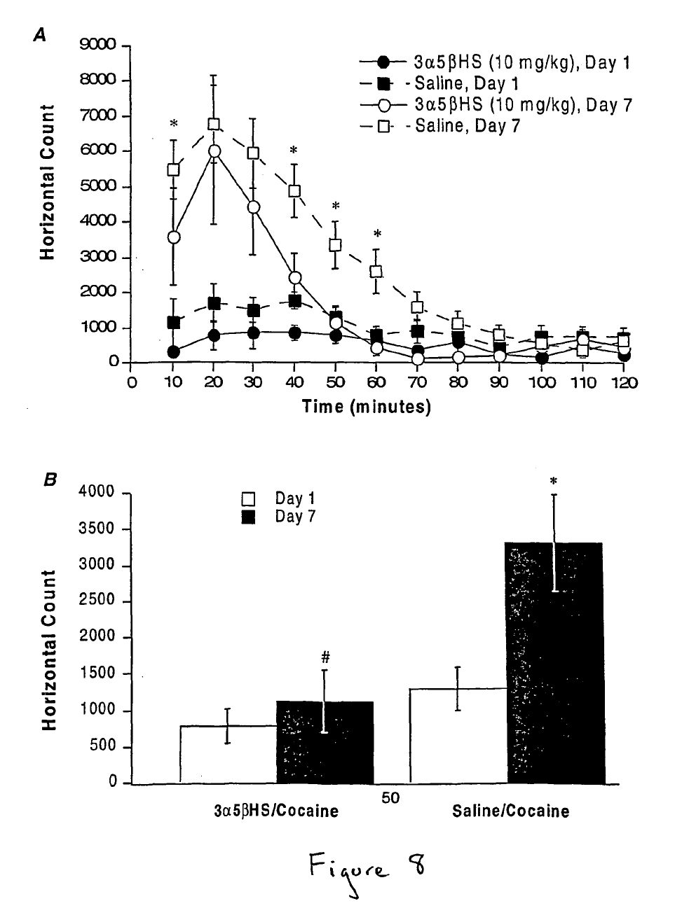

The data summarized in FIG. 8 indicate that repeated cocaine injections result in sensitization of the behavioral response to cocaine in that behavioral response elicited on day 7 was significantly greater than on day 1 in the saline/cocaine group. Administration of 3α5βHS (10 mg/kg) prior to each of 7 daily injections of cocaine inhibited the development of behavioral sensitization to cocaine compared to control (FIG. 8). The complete time course of the sensitization data are presented in FIG. 8A. The data were analyzed separately for day 1 and day 7 with a mixed factors ANOVA with repeated measures over time. The analyses revealed a significant main effect of time for day 1 [F(11,110)=2.59; p<0.0057] and day 7 [F(11,110)=25.0; p<0.0001]. Subsequent pairwise analyses (Fisher's LSD) revealed significant differences between the day 7 saline/cocaine and 3α5βHS/cocaine groups at 10, 40, 50 and 60 min following the injections. FIG. 8B represents the data from FIG. 8A at 50 min post injection. The data were analyzed by one-way ANOVA, followed by Fisher's LSD. There were significant main effects of treatment [F(1,20)=7.871, p<0.0109] and days of treatment [F(1,20)=6.009, p<0.0235]. While there was only a marginally significant treatment×time interaction [F(1,20)=3.023, p<0.0975], post-hoc analyses (Fisher's LSD) revealed a significant increase in horizontal activity on day 7 in the saline/cocaine group as compared to day 1 saline/cocaine indicating cocaine-induced behavioral sensitization. Furthermore, the analyses also revealed a significant difference between the day 7 3α5βHS/cocaine and saline/cocaine groups, suggesting that 3α5βHS (10 mg/kg) inhibits cocaine induced behavioral sensitization in rats (FIG. 8B).

Behavioral sensitization is a progressive and enduring augmentation of locomotor and stereotyped behavior in rats resulting from intermittent injections of cocaine and related psychostimulants. Recent self-administration experiments indicate that psychostimulant sensitization increases the reinforcing efficacy of these drugs. Rats sensitized to psychostimulants subsequently self-administer subthreshold doses of cocaine or amphetamine (Horger et al., 1990; Vezina et al., 1999) and display a higher break point for cocaine or amphetamine self-administration using a progressive ratio schedule of reinforcement (Mendrek et al., 1998; Lorrain et al., 2000). Pretreatment with systemic injections of 3α5βHS, a negative modulator of the NMDA receptor, attenuated the development of behavioral sensitization to cocaine.

The results show that 3α5βHS appears in the brain immediately following peripheral injections. Behavioral experiments indicate that 3α5βHS inhibits NMDA-induced increases in motor activity and impair the development of behavioral sensitization to cocaine. Neurochemical experiments show that 3α5βHS, but not its pregnane metabolites, block NMDA-induced increases in dopamine release in the rat striatum. These findings suggest that 3α5βHS negatively modulates NMDA receptors in vivo.

### Example 6

### 3α5βHS Attenuates NMDA-induced Behavioral Activation

The ability of 3α5βHS to dose-dependently decrease the behavioral responses elicited by intra-accumbal microinjections of NMDA is consistent with in vitro studies showing that 3α5βHS inhibits NMDA receptors (Park-Chung et al., 1994) and studies showing that excitatory amino acids regulate striatal-mediated motor activity in that NMDA infused into the striatum resulted in an increase in behavioral activation (Riederer et al., 1992; Schmidt et al., 1992; Ossowska and Wolfarth, 1995). The attenuation of NMDA-induced behavioral hyperactivity by 3α5βHS does not appear to be due to a sedative effect of the steroid since the highest used had no effect on the non-pharmacological behavioral activation induced by saline administration or on the behavioral hyperactivity induced by an acute cocaine injection.

Administration of NMDA at concentrations of 1 mM or higher into the striatum increases extracellular levels of dopamine (Taber et al., 1996; Ohno et al., 1995; (Cheramy et al., 1986; Moghaddam et al., 1990; Keefe et al., 1992) and the concentration of NMDA microinjected in our behavioral experiment (0.1 μg/0.5 μl) was slightly higher than 1 mM. Since increases in extracellular dopamine in the striatum are well known to increase motor activity (Wu et al., 1993; Taepavarapruk et al., 2000), we hypothesize that the increase in motor activity seen in rats receiving striatal microinjections of NMDA is secondary to increased NMDA-induced dopamine release and that 3α5βHS inhibits this motor activity via negative modulation of the NMDA receptor.

### Example 7

### 3α5βHS Blocks NMDA-induced Dopamine Release in the Striatum

Administration of NMDA into the striatum increases extracellular levels of dopamine both in vitro (Roberts and Sharif, 1978; Roberts and Anderson, 1979; Marien et al., 1983) and in vivo (Cheramy et al., 1986; Moghaddam et al., 1990; Keefe et al., 1992) and NMDA antagonists block the enhanced dopamine release (Marek et al., 1992; Whitton et al., 1994). Consistent with these findings, in the present studies NMDA administered into the striatum through the microdialysis probe increased extracellular dopamine levels. Moreover, the competitive NMDA antagonist, D-AP5, dose-dependently reduced the NMDA-induced increase in striatal extracellular dopamine.

Our results also indicate that the systemic injection of 3α5βHS decreased NMDA-induced dopamine release in a dose-dependent manner. The effect of 3α5βHS on NMDA-induced dopamine release was similar to that of the NMDA antagonist, D-AP5, which is consistent with its action as a negative modulator of the NMDA receptor in vitro. The effects of 3α5βHS and D-AP5 on striatal dopamine release are presumably due to an action on NMDA receptors located presynaptically on dopaminergic nerve terminals. These results indicate that NMDA evokes dopamine release in the striatum by stimulating NMDA receptors on dopaminergic nerve terminals, which is presumably the site of action of the negative NMDA modulator, 3α5βHS.

### Example 8

In order to address the question of whether 3α5βHS or its main metabolite, pregnanolone, was responsible for the observed pharmacological and behavioral effects, a sub-sedative dose of pregnanolone was used in microdialysis experiments. This dose of pregnanolone had no effect on NMDA-induced striatal dopamine release, indicating that neither pregnanolone nor a pregnanolone metabolite is likely to be responsible for the inhibition of NMDA-induced dopamine release produced by 3α5βHS.

### Example 9

### 3α5βHS is Antinociceptive in a Dose-dependent Manner

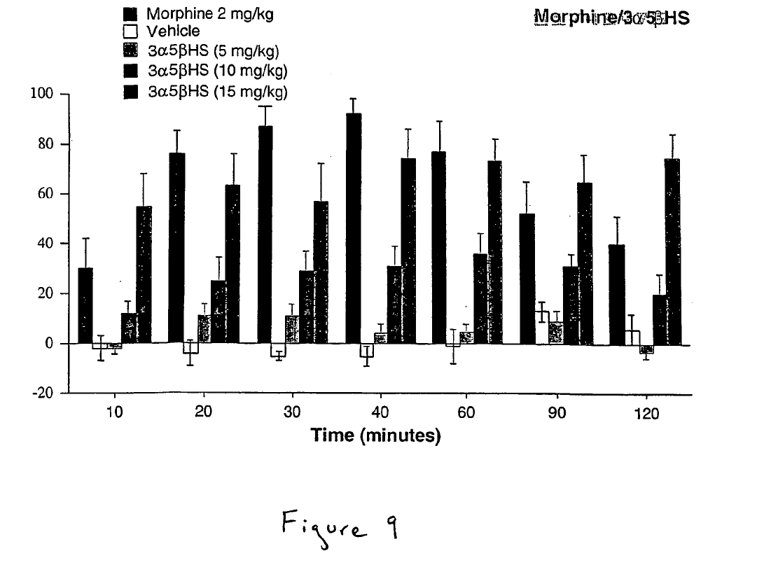

As shown in FIG. 9, morphine (2 mg/kg, s.c.) and 3α5βHS (5, 10 and 15 mg/kg i.p.) elicit a dose-dependent increase in tail flick latency. At the highest doses, 10 and 15 mg/kg, 3α5βHS, induced near maximal lengthening of the latency time, close to the cut-off time value (10 seconds). While this effect is comparable to the analgesic effect of morphine (2 mg/kg, s.c.) it is longer lasting. Morphine is most effective 20 minutes following injection but its effect is short lived compared to the analgesic effect of the steroid. The data were analyzed using a one-way ANOVA followed by Fisher's LSD. The analysis revealed a significant effect of time [F(6,24)=3.605, p<0.0018], treatment [F(4,24)=99.274, p<0.0001] and time×treatment interaction [F(24,287)=1.79, p<0.0146]. Subsequent pair-wise analysis revealed significant levels of analgesia for 3α5βHS (10 and 15 mg/kg) and morphine (2 mg/kg) groups compared to control at 20, 30, 40, 60, 90 min. 3α5βHS (15 mg/kg) was also effective in increasing the latency compared to the other groups at 90 and 120 min.

### Example 10

### PACME is Antinociceptive in a Dose-dependent Manner

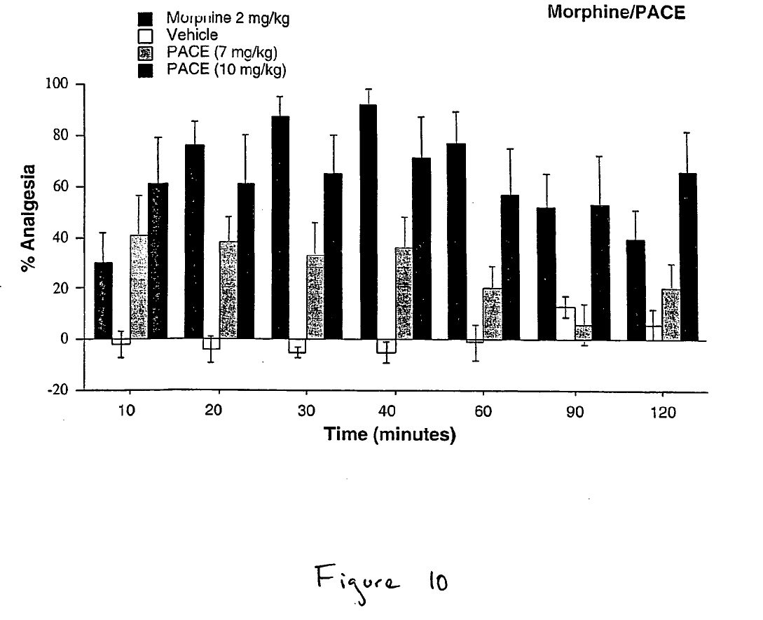

PACME produced analgesia similar to that of morphine in a dose-dependent manner compared to control (FIG. 10). PACME, induced near maximal lengthening of the latency time, close to the cut-off time value (10 seconds). While this effect is comparable to the analgesic effect of morphine (2 mg/kg, s.c.) it is longer lasting. The data were analyzed using a one-way ANOVA followed by Fisher's LSD. The analysis revealed a significant effect of time [F(6,30)=3.142, p<0.0053], and treatment [F(5,30)=63.355, p<0.0001]. Subsequent pair-wise analysis revealed significant levels of analgesia for PACME (7 and 10 mg/kg) and morphine (2 mg/kg) groups compared to control at 20, 30, 40, 60 min following injection. Furthermore, PACME (10 mg/kg) increased the latency to remove the tail form the noxious light stimulus at 90 and 120 min.

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating neurologic disease or disease symptom in a subject comprising administration of an effective amount of a neuroactive steroid.

2. The method of claim 1, wherein the neurologic disease or disease symptom is drug addiction.

3. The method of claim 1, wherein the neurologic disease or disease symptom is schizophrenia.

4. The method of claim 1, wherein the neurologic disease or disease symptom is Parkinson's disease.

5. The method of claim 1, wherein the neuroactive steroid crosses the blood-brain barrier.

6. The method of claim 1, wherein the neuoractive steroid is pregnanolone hemisuccinate.

7. The method of claim 1, wherein the neurologic disease or disease symptom is mediated by NMDA.

8. The method of claim 1, wherein the neurologic disease or disease symptom is mediated by NMDA-induced increase in dopamine release.

9. The method of claim 1, wherein the neurologic disease or disease symptom is mediated by glutamatergic transmission.

10. The method of claim 9, wherein the neurologic disease or disease symptom involves negative modulation of glutamatergic transmission.

11. The method of claim 1, wherein the neurologic disease or disease symptom is treated by cognitive enhancement.

12. The method of claim 11, wherein the neuoractive steroid affects an impaired subject but essentially does not affect a normal subject.

13. The method of claim 11, wherein the neuoractive steroid is pregnenolone hemisuccinate.

14. The method of claim 11, wherein the neurologic disease or disease symptom is amnesia.

15. A method of inducing acute anesthesia in a subject comprising administration of an effective amount of a neuroactive steroid.

16. A method of treating a disorder, the disorder being characterized by excessive NMDA levels in a subject, comprising administration of an effective amount of a neuroactive steroid.

17. The method of claim 16, wherein the neuroactive steroid is pregnanolone hemisuccinate.

18. A method of treating a disorder potentiated by NMDA in a subject comprising administration of an effective amount of a neuroactive steroid.

19. The method of claim 18 wherein the neuroactive steroid is pregnenolone hemisuccinate.

20. A method of enhancing memory function in a subject comprising administration of an effective amount of a neuroactive steroid.

21. The method of claim 20, wherein the neuroactive steroid is pregnenolone hemisuccinate.

22. A method of enhancing acetylcholine release in the brain of a subject comprising administration of an effective amount of a neuroactive steroid.

23. The method of claim 22, wherein the neuroactive steroid is pregnenolone hemisuccinate.
